(19) United States
(12) Patent Application Publication
(10) Pub. No.: US 2024/0351070 A1
(43) Pub. Date: Oct. 24, 2024

(54) ROW-COLUMN ADDRESSING ULTRASOUND TRANSDUCTION DEVICE

(71) Applicant: VERMON SA, Tours (FR)

(72) Inventors: Mathieu LEGROS, Tours (FR); Stéphane LAFON, Tours (FR)

(21) Appl. No.: 18/608,079

(22) Filed: Mar. 18, 2024

(30) Foreign Application Priority Data
Apr. 24, 2023 (FR) 23/04069

(51) Int. Cl. B06B 1/06; A61B 8/00; H10N 30/06; H10N 30/088

(52) U.S. Cl. CPC B06B 1/0629; B06B 1/067; H10N 30/06; H10N 30/088; A61B 8/4488; B06B 2201/76

(57) ABSTRACT

A row-column addressing array ultrasound transduction device including a metallized plate of active material comprising a plate of active material having a first main surface and a second main surface opposite to the first main surface, a first metallic main surface layer on the first main surface, and a second metallic main surface layer on the second main surface.

### FIELD OF THE INVENTION

The present disclosure concerns ultrasound transduction devices comprising an array of elementary ultrasound transducers electrically connected by arrays of electrodes in rows and in columns called row-column addressing ultrasound transduction devices or RCA ultrasound transduction devices.

### DESCRIPTION OF RELATED ART

Row-column addressing ultrasound transduction devices have become more and more attractive in the field of ultrasound imaging since they allow a simultaneous imaging in at least two planes, and may also be used for a 3D volume imaging.

One of the main advantages of RCA transducers lies in the number of electronic circuits necessary to drive the transducer elements. For an array formed of R rows and C columns, R+C drive channels are typically needed in the case of an RCA array while typically R×C channels are necessary for a fully populated array, in other words for which each elementary transducer is individually connected. In an application where R=C, an electronic driver system typically having 64, 128, or 256 channels may drive an RCA device having a number of elements respectively of 32×32, 64×64, or 128×128, while for a fully populated ultrasound transduction device, the maximum number of elementary transducers will respectively be limited to 8×8, 11×11, or 16×16.

For applications where the electronic circuits for driving the transducers are offset from the probe comprising the array of ultrasound transducers, an RCA array advantageously enables to decrease the number of coaxial cables between the probe and the offset electronic system. Alternatively, the decrease in the number of channels advantageously enables to integrate the electronic driver circuits in the probe at closest to the array of elementary transducers. For applications where interconnects of printed circuit board (PCB) or flex printed circuit board type are used to connect the transducers to the coaxial cables or to electronic driver circuits embedded at closest to the transducers, RCA transducers advantageously decrease the number of interconnects and accordingly enable to have less complex PCBs by decreasing the number of necessary PCB layers as well as the number of connections between layers (vias). Thinner PCBs decrease the impact on the acoustic propagation, but, as will be discussed hereafter, the impedance mismatch caused by the materials of the PCBs however remains a problem.

FIG. 1 illustrates an example of an RCA transducer 10, comprising a layer of active material 12 (for example, a piezoelectric, piezocomposite, or monocrystalline material), a row interconnection array 14 (R), a column interconnection array 16 (C), an acoustic impedance matching layer 18, and a back side layer 20. The main surfaces of active material 12 are fully or partially metallized in the areas in front of the column and row arrays 16 and 14 (not shown in the drawing). Acoustic impedance matching layer 18 is a layer or layers of a material having an acoustic impedance with a value between the acoustic impedance of the layer of active material 12 and the acoustic impedance of an object being imaged (for example, a human body) to optimize the transmission of the acoustic wave between the two mediums. The main outer surface of the impedance matching layer thus forms the output surface for the acoustic waves emitted by the transducer. Back side layer 20 is a layer or a multilayer of one or a plurality of acoustically-absorbing materials to avoid parasitic reflections of acoustic waves. By convention, row interconnection array 14 (R) is located between the layer of active material 12 and the layer of back side material 20, and column interconnection array 16 (C) is located between the layer of active material 12 and the layer of acoustic material 18.

However, the presence of interconnection materials, for example comprising electrically-insulating materials such as polyimide and electrically-conductive materials such as copper, may negatively affect the performance of RCA ultrasound transduction device 10, as will be discussed hereafter, and the manufacturing/the assembly of the conventional interconnection structure is complicated by the fact that the two arrays of electrodes (electrodes of array R & electrodes of array C) have to be interconnected on opposite main surfaces of the layer of active material 12.

Concerning the interconnection materials, RCA ultrasound transduction device 10 requires at least the interconnection array 16 (C) between the layer of active material 12 and acoustic impedance matching layer 18. Certain disadvantages of interconnection array 16 (C) include an impedance mismatch between the layer of active material 12, interconnection array 16 (C), and acoustic impedance matching layer 18, and a low uniformity between elements. Concerning the impedance mismatch, interconnection array 16 (C) comprises an insulating material of low acoustic impedance, typically 3 MRayl, as compared with the layer of active material 12, typically of 30 MRayl, and, accordingly, creates a significant acoustic impedance mismatch between the layer of active material 12 and acoustic matching layer 18. A possible solution could be for the interconnection layer to only partially extend above the metallized surfaces of the column array. Typically, the interconnection layer covers one (or both) end(s) of the metallized surface extensions of the column array and extends along these metallized surfaces above a few lateral elements of the row array. In such a configuration, the height of the interconnection layer creates a space between the metallized surfaces of the column array and the layer of acoustic material 12. Even if the space can be filled with a material, in terms of uniformity, the acoustic properties are accordingly not identical over the entire surface of the transducer, according to the length of the extension of the interconnection layer over the metallized surfaces of the column array, the acoustic load, that is, the impedance seen by part of the elements, is not the same. In terms of quality of the acoustic propagation, it is always desirable to have a direct contact between the layer of active material 12 and acoustic matching layer 18, or if this is not possible, to have an adhesive material as thin as possible interposed therebetween.

### BRIEF SUMMARY OF THE INVENTION

An embodiment provides a row-column addressing array ultrasound transduction device, comprising:
a metallized plate of active material comprising a plate of active material having a first main surface and a second main surface opposite to the first main surface, a first metallic main surface layer on the first main surface, and a second metallic main surface layer on the second main surface;
an acoustic impedance matching layer bonded to the second metallic main surface layer;
a first set of parallel cutting notches oriented in a first direction, said notches of the first set extending across the entire thickness of the metallized plate of active material and along the entire length of the metallized plate of active material in the first direction, and individualizing the second metallic main surface layer into a set of column electrodes;
a second set of parallel cutting notches oriented along a second direction different from the first direction, said notches of the second set extending across the entire thickness of the first metallic main surface layer and across at least part of the thickness of the plate of active material, and along the entire length of the metallized plate of active material in the second direction, and individualizing the first metallic main surface layer into a set of rows of row electrodes and a first external row of column electrode contacts, each column electrode contact of the first external row of column electrode contacts being electrically connected to a respective column electrode of the set of column electrodes; and
an interconnection layer comprising a substrate of an electrically-insulating material, a first set of conductive tracks on the substrate, each track of the first set of conductive tracks being in electrical contact with a respective column electrode contact of the first external row of column electrode contacts, and a second set of conductive tracks on the substrate, each track of the second set of conductive tracks being in electrical communication with a respective row among the rows of row electrodes.

According to an embodiment, each column electrode contact of the first external row of column electrode contacts is electrically connected to the corresponding respective column electrode of the set of column electrodes by a respective column electrode connector of a first set of column electrode connectors formed in a first lateral surface metal layer covering a first lateral surface of the plate of active material, the column electrode connectors of the first set of column electrode connectors being individualized by the cutting notches of the first set of cutting notches.

According to an embodiment, each column electrode contact of the first external row of column electrode contacts is electrically connected to the corresponding respective column electrode of the set of column electrodes by a metallized via vertically crossing the plate of active material.

According to an embodiment, the substrate of the interconnection layer comprises an inner surface, the first set of conductive tracks and the second set of conductive tracks are on the inner surface of the substrate, and the first set of conductive tracks forms a first interconnect fan-out of the interconnection layer, and the second set of conductive tracks forms a second interconnect fan-out of the interconnection layer.

According to an embodiment, the substrate of the interconnection layer comprises an inner surface, an outer surface, vias respectively aligned with the first external row of column electrode contacts, and respective via connectors in the vias, the first set of conductive tracks is on the outer surface of the substrate and is in electrical communication with respective column electrode contacts of the first external row of column electrode contacts by means of via connectors, and the second set of conductive tracks is on the inner surface of substrate, and the first set of conductive tracks and the second set of conductive tracks form a first interconnect fan-out of the interconnection layer.

According to an embodiment, the second set of parallel cutting notches further individualizes a second external row of column electrode contacts, each column electrode contact of the first external row of column electrode contacts being electrically connected to a respective column electrode of the set of column electrodes.

According to an embodiment, the first and second directions are orthogonal.

According to an embodiment, the first set of cutting notches and the second set of cutting notches are filled with a polymer material.

According to an embodiment, the interconnection layer is a flex printed circuit board.

According to an embodiment, the device further comprises a set of backing layers bonded to the interconnection layer.

Another embodiment provides a method of manufacturing a row-column addressing array ultrasound transduction device, the method comprising:
the provision of a raw metallized plate of active material comprising a plate of active material having a first main surface and a second main surface opposite to the first main surface, a first metallic main surface layer on the first main surface, and a second metallic main surface layer on the second main surface, the metallized raw plate of active material having a width Wp and a length Lp;
the cutting of the metallized raw plate of active material to individualize a metallized plate of active material from the metallized raw plate of active material;
the bonding of an acoustic impedance matching layer to the second metallic main surface layer of the metallized plate of active material;
the full cutting of the metallized plate of active material in a first direction to form a first set of parallel cutting notches, the first set of cutting notches forming in the second metallic main surface layer a set of column electrodes; the partial cutting of the metallized plate of active material in a second direction to form a second set of parallel cutting notches, the second set of cutting notches forming in the first metallic main surface layer rows of row electrodes and a first external row of column electrode contacts, each column electrode contact of the first external row of column electrode contacts being electrically connected to a respective column electrode of the set of column electrodes; the provision of an interconnection layer comprising a substrate of an electrically-insulating material, a first set of conductive tracks on the electrically-insulating substrate, a second set of conductive tracks on the electrically-insulating substrate;
the connection of each track of the first set of conductive tracks so that it is in electrical communication with a respective column electrode contact of the first external row of column electrode contacts; and the connection of each track of the second set of conductive tracks in electrical contact with a respective row among the rows of row electrodes.

According to an embodiment, each column electrode contact of the first external row of column electrode contacts is electrically connected to the corresponding respective column electrode of the set of column electrodes by a respective column electrode connector of a first set of column electrode connectors formed in a first lateral surface metal layer of the metallized plate of active material, covering a first lateral surface of the plate of active material, the column electrode connectors of the first set of column electrode connectors being individualized by the cutting notches of the first set of cutting notches.

According to an embodiment, the metallized plate of active material has a length La smaller than the length Lp of the metallized raw plate of active material and a width Wa equal to the width Wp of the metallized raw plate of active material.

According to an embodiment, each column electrode contact of the first external row of column electrode contacts is electrically connected to the corresponding respective column electrode of the set of column electrodes by a metallized via of the metallized plate of active material, vertically crossing the plate of active material.

According to an embodiment, the metallized plate of active material has a length La smaller than the length Lp of the metallized raw plate of active material and a width Wa smaller than the width Wp of the metallized raw plate of active material.

### BRIEF DESCRIPTION OF THE DRAWINGS

Embodiments will be described hereafter in relation with the accompanying drawings and examples will be provided as an illustration without for all this limiting the scope of the claims:

FIG. 1 is a simplified diagram of an exploded conventional interconnection structure of an RCA ultrasound transduction device.

FIG. 2 is a perspective view of a raw plate of active material according to the invention.

FIG. 3 is a perspective view of the raw plate of active material of FIG. 2 after a metallization for forming a metallized plate of active material.

FIG. 4 is a perspective view of a metallized plate of active material resulting from the cutting of the metallized raw plate of active material of FIG. 3.

FIG. 5 is a perspective view of the metallized plate of active material of FIG. 4 bonded to an acoustic impedance matching layer to form an intermediate assembly.

FIG. 6 is a perspective view of the intermediate assembly of FIG. 5 after the full cutting of the metallized plate of active material in a first direction to form a first set of parallel kerfs.

FIG. 7 is a perspective view of the intermediate assembly of FIG. 6 after the partial cutting of the metallized plate of active material in a second direction to form a second set of parallel kerfs.

FIG. 8 is a perspective view of the intermediate assembly of FIG. 7 after the bonding of an interconnection layer.

FIG. 9 is an enlarged view of a corresponding portion of the intermediate assembly of FIG. 8.

FIG. 10 is a perspective view of a transducer in an array of rows and columns assembled according to the invention.

FIG. 11 is a perspective view of the intermediate assembly after the partial cutting of a metallized plate of active material to create kerfs, where the kerfs are filled with a polymer material according to an alternative embodiment of the invention.

FIG. 12 is a perspective view of the intermediate assembly of FIG. 11 after the bonding of an interconnection layer.

FIG. 13 is a perspective view of an intermediate assembly according to another embodiment of the invention, where a first set of conductive tracks is located on an outer surface of a substrate of an interconnection layer and a second set of conductive tracks is located on an inner surface of the substrate of the interconnection layer.

FIG. 14 is a perspective view of a plate of active material after metallization according to another embodiment.

FIG. 15 is a perspective view of a metallized plate of active material resulting from the cutting of the metallized plate of active material of FIG. 14.

FIG. 16 is a perspective view of an intermediate assembly after the partial cutting of the metallized plate of active material of FIG. 15 in first and second directions.

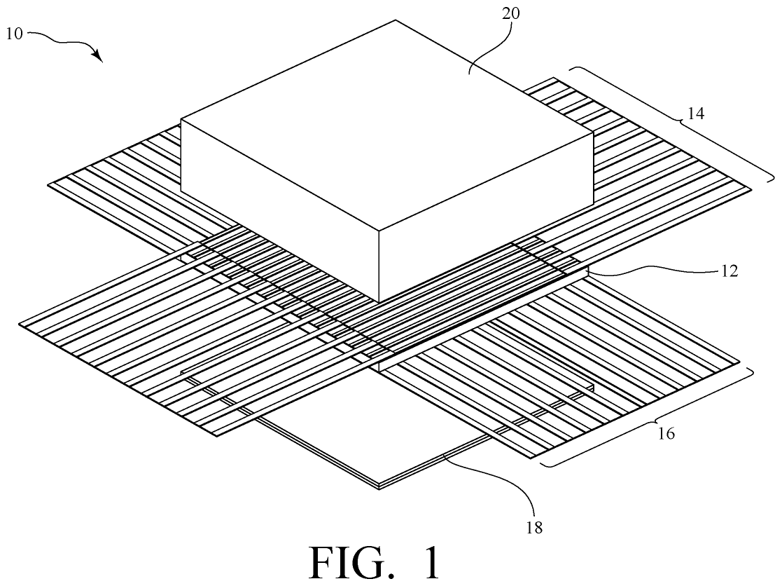

### DETAILED DESCRIPTION OF THE INVENTION

The details of one or of a plurality of embodiments of the object described herein are disclosed in this document. Modifications to the embodiments described in this document, as well as other embodiments, will readily occur to those skilled in the art after studying the information disclosed in this document. The information disclosed in this document and, in particular, the specific details of the described examples of embodiment, are essentially provided for clarity and to make the understanding easier and are not limiting. In case of a conflict, the description of this document, including the definitions, will hold the priority.

Although the following terms are supposed to be well known by those skilled in the art, definitions are given to facilitate the explanation of the object described herein.

Unless specified otherwise, all the technical and scientific terms used herein have the same meaning as that commonly understood by those skilled in the art in the concerned field. Similarly, the methods, devices, and materials described herein allow the implementation or the testing of the present invention, although other methods, devices, and materials similar or equivalent to those described herein may be use to achieve the same purposes.

The terms “one” and “the” designate “one or a plurality of” when they are used in this application, including in the claims. Thus, for example, the reference to “a transducer” includes a plurality of such transducers and so on.

Unless specified otherwise, all the numbers expressing composition components, properties such as frequency, etc., used in the disclosure and the claims are to be understood as being in all circumstances modified by the term “approximately”. Accordingly, unless specified otherwise, the numerical parameters indicated in these description and claims are approximations that may vary according to the desired properties which are desired to be obtained by the object described herein.

As used herein, the term “approximately”, when it is affected to a value or to a quantity, is supposed to incorporate variations of ±20% in certain embodiments, of ±10% in certain embodiments, of ±5% in certain embodiments, of ±1% in certain embodiments, of ±0.5% in certain embodiments, and of ±0.1% in certain embodiments with respect to the indicated quantity, since such variations are appropriate to implement the described object.

As used herein, ranges can be expressed as starting “from approximately” a specific value, and/or to “approximately” another specific value. It will also be understood that there is a number of values described herein, and that each value is also described herein as being “approximately” this specific value in addition to the actual value. For example, if value “10” is described, then “approximately 10” is also described. It will also be understood that each unit between two specific units is also described. For example, if 10 and 15 are described, then 11, 12, 13, and 14 are also described.

The terms “transducer array” or “transducer network” or “ultrasound transduction device” are used herein to described a transducer device obtained by a geometric arrangement of a plurality of transducers (that is, of transducers element) having dimensions compatible with the focusing and deflection characteristics of an ultrasound beam.

The terms “elementary transducer” or “transducer element” or “transducer” are used herein to describe an individual ultrasound transducer component of a transducer array. Generally, an elementary transducer of a transducer array has planar dimensions appropriate for an electronic focusing and deflection of ultrasound beams. Each transducer element or elementary transducer is provided with two conductive electrodes. A conductive electrode may be either formed by subtractive and additive methods in the case of an array of elements without cutting, or etched at the same time as the layer of active material during the process of individualization of the elements.

A row-column addressing (RCA) ultrasound transduction device, as previously disclosed, is an array transducer having “R” rows of electrodes and “C” columns of electrodes on opposite sides of an active material, where a row interconnection array R comprises row tracks which are connected to the rows on a first main surface of the array of transducers and a column interconnection array C is connected to the columns on a second main surface of the array opposite to the first surface. Individual transducer elements are actuated by applying appropriate signals on a row track of row interconnection array R and on a column track of column interconnection array C corresponding to the transducer element being activated.

As used herein, the term “active material” signifies an ultrasound transducer material which undergoes compressive and stretching efforts to convert electrical energy into ultrasounds or conversely. Examples of such active materials include a piezoelectric material such as PZT, KNN, BaTiO3, PMN-PT, LNO3 either in the form of a ceramic or monocrystalline material, or forming the entire bulk of the active material, or in composite form (mixed to a polymer material), as well as a piezoelectric polymer such as PVDF. A “plate of active material” is a thin and flat sheet of an active material. A “raw plate of active material” is a large thin and flat sheet of an active material from which smaller plates of the active material are cut or individualized in another way.

As used herein, the term “acoustic impedance matching layer” or “matching layer” signifies a layer or layers of a material bonded on a front side of the active material of the ultrasound transducer having an acoustic impedance with a value between that of the acoustic impedance of the active material and that of the acoustic impedance of the element to be imaged (for example human tissue). This impedance matching layer enables to minimize the reflection of the ultrasound wave at the interface between the active material and the medium to be imaged, for example a patient's body.

As used herein, the term “backing material” or “back-side layer” signifies a layer or layers of one or a plurality of acoustic damping materials bonded to a back side of the active material of the ultrasound transducer to avoid parasitic reflections of acoustic waves.

As used herein, the terms “cutting” signify cutting a material or a plurality of materials assembled together to former trenches or kerfs in the active material or to individualize a plate of active material from a raw plate. Preferred cutting means are a rotation circular diamond wire saw. “Cutting” may also include laser cutting or chemical etching as cutting means.

As used herein, the term “kerf” signifies a slot or a notch formed by cutting, for example with a saw or other cutting means (see “cutting”).

As used herein, the term “C direction” signifies a column direction of the array of elementary transducers.

As used herein, the term “R direction” signifies a row direction of the array of elementary transducers.

As used herein, the term “interconnection layer” signifies a layer of insulating material having electric tracks formed thereon to interconnect electrodes of individual elements of an RCA transduction device with an imaging system.

As used herein, the term “fan-out” or “interconnect fan-out” designates the electrical interconnects between the transducer elements and the upstream electrical connections in order to adapt the pitch and the direction of the electrical contacts at each end of the interconnect. The interconnect fan-out is for example obtained with a flex printed circuit board.

As used herein, the term “anisotropic conductive film” signifies an adhesive interconnect system for forming electrical and mechanical connections from electronic control circuits to substrates.

The term “imaging system” is here used to described a device which comprises a signal generator, a signal processor, and a user interface. The signal generator is intended to generate signals for activating transducer elements of an ultrasound transducer to generate acoustic waves. The signal processor is intended to process the signals generated by the transducer elements as a response to acoustic waves received by the transducer elements. The signal processor further comprises an image processor intended to manage images based on the processed signals. The user interface is intended to display the generated images and to receive additional inputs from a user of the imaging system. Since such imaging systems are available for sale and known by those skilled in the art, the elements and the operation of such imaging systems will not be described any further herein.

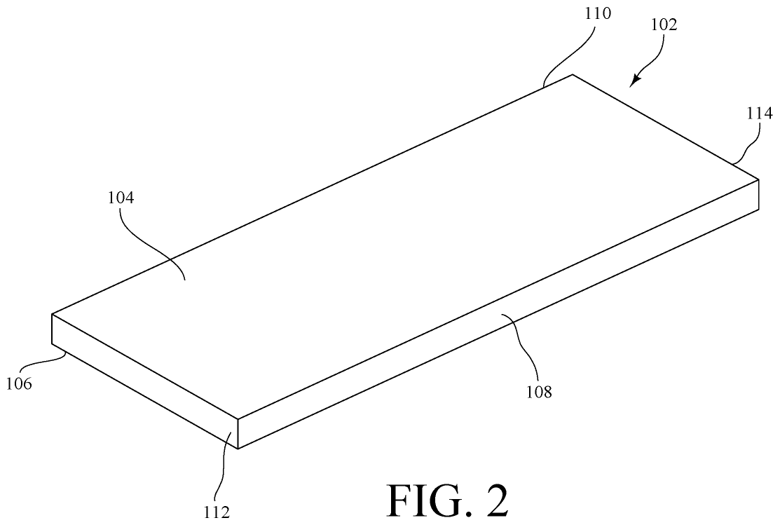

A first example of a method of manufacturing an ultrasound transducer in an array of rows and columns according to the invention starts by the provision, as illustrated in FIG. 2, of a raw plate of active material 102 having a first main surface 104 and a second main surface 106 opposite to first main surface 104. On sides extending between first main surface 104 and second main surface 106, the raw plate of active material 102 also has a first lateral surface 108, a second lateral surface 110 opposite or facing the first lateral surface 108, a third lateral surface 112, and a fourth lateral surface 114 opposite or facing the third lateral surface 112.

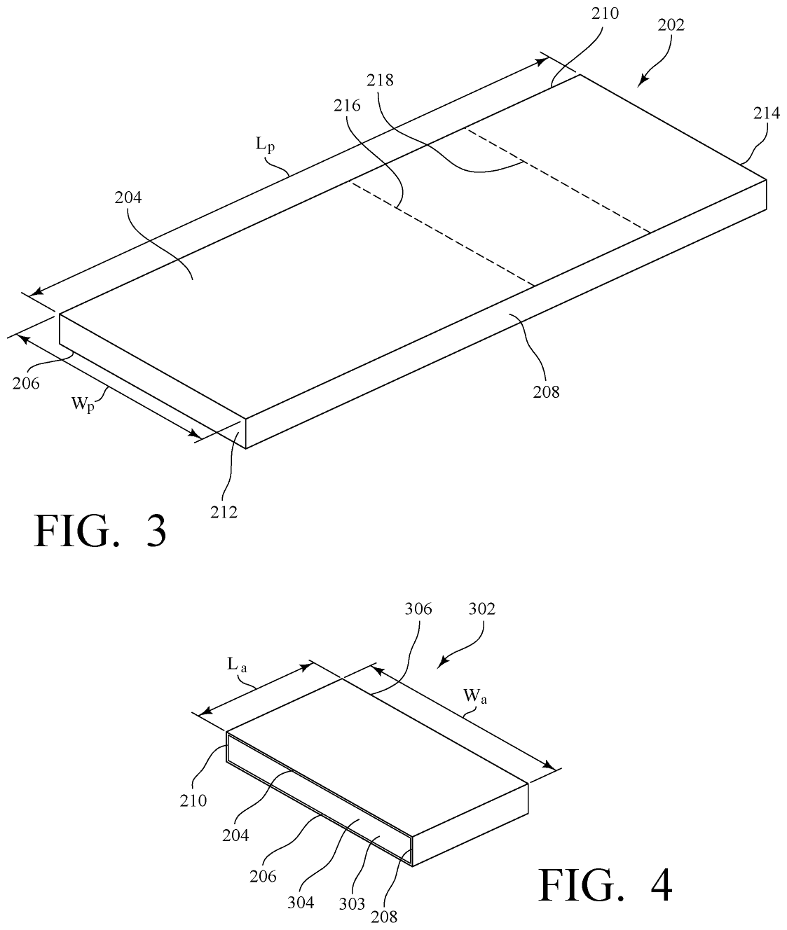

The raw plate of active material 102 is then metallized to form a metallized raw plate of active material 202, as shown in FIG. 3. There appear in FIG. 3 a first metallic main surface layer 204, a second metallic main surface layer 206, a first lateral surface metal layer 208, a second lateral surface metal layer 210, a third lateral surface metal layer 212, and a fourth lateral surface metal layer 214. As compared with the raw plate of active material 102 (FIG. 2), the first metallic main surface layer 204 is on the first main surface 104, the second metallic main surface layer 206 is on the second main surface 106, the first lateral surface metal layer 208 is on the first lateral surface 108, the second lateral surface metal layer 210 is on the second lateral surface 110, the third lateral surface metal layer 212 is on the third lateral surface 112, and the fourth lateral surface metal layer 214 is on the fourth lateral surface 114. The first lateral surface metal layer 208, the second lateral surface metal layer 210, the third lateral surface metal layer 212, and the fourth lateral surface metal layer 214 are electrically connected to the first metallic main surface layer 204 and to the second metallic main surface layer 206. This is designated hereafter as being a “wrap-around” metallization. The raw plate of active material 102 (FIG. 2) is accordingly fully metallized, although a single one of the first lateral surface metal layer 208 and of the second lateral surface metal layer 210 is necessary to provide a single interconnection layer for both the R array electrodes and the C array electrodes, as will be discussed hereafter. The metallized raw plate of active material 202 has a width, Wp, and a length, Lp.

An example of a method of metallization of a plate of active material comprises the deposition of one or of a plurality of layers of electrically-conductive materials, for example gold (Au) or copper (Cu), nickel (Ni) or silver (Ag). The deposition method is for example obtained by physical vapor deposition (PVD) or by chemical vapor deposition (CVD). Thus, the first metallic main surface layer 204, the second metallic main surface layer 206, and the lateral surface metal layers 208, 210, 212, 214 are very thin layers ranging from a few tens (for example, from 20 to 40) of nanometers to a few (for example, from 2 to 4) micrometers to be compared with the dimensions of the raw plate of active material having a thickness in the range from a few tens (for example, from 20 to 40) of micrometers to a few (for example, from 2 to 4) millimeters and lateral dimensions ranging from a few (for example, from 2 to 4) millimeters to some hundred millimeters. Thus, even though the raw plate of active material 102 is under the first metallic main surface layer 204, the second metallic main surface layer 206, and the lateral surface metallic layers 208, 210, 212, 214 and is accordingly not visible in FIG. 3, it will be understood by those skilled in the art that the width, length, and thickness of the raw plate of active material 102 are substantially the same as the width, Wp, length, Lp, and thickness of the metallized raw plate of active material 202.

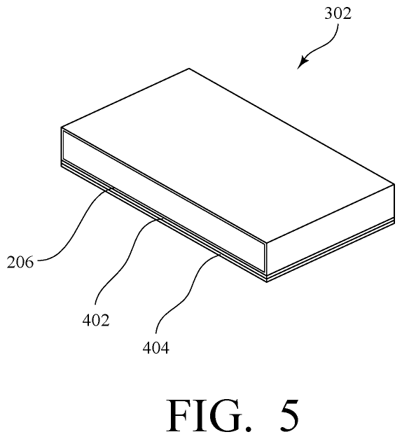

The first example of a method of manufacturing an ultrasound transducer according to the invention then comprises a step of cutting of the metallized raw plate of active material 202 widthwise at two cutting locations 216, 218 to individualize a metallized plate of active material 302, as shown in FIG. 4, from the metallized raw plate of active material 202. The metallized plate of active material 302 has a width Wa equal to Wp, a length La smaller than length Lp, and a thickness equal to the thickness of the metallized raw plate of active material 202. Length La is equal to the final length of ultrasound transduction device RCA.

At the center of the metallized plate of active material 302 is located a plate of active material 303 individualized from the raw plate of active material 102 (FIG. 2). The plate of active material 303 has a first main surface corresponding to the first main surface 104 of the raw plate of active material 102, a second main surface opposite to the first main surface and corresponding to the second main surface 106 of the raw plate of active material 102, a first lateral surface extending between the first main surface and the second main surface and corresponding to the first lateral surface 108 of the raw plate of active material 102, and a second lateral surface corresponding to the second lateral surface 110 of the raw plate of active material 102. The thickness of the plate of active material 303 (that is, the thickness of the plate of active material) corresponds to the thickness of the raw plate of active material. The first metallic main surface layer 204, the second metallic main surface layer 206, the first lateral surface metal layer 208, and the second lateral surface metal layer 210 cover the first main surface, the second main surface, the first lateral surface, and the second lateral surface, respectively, of the plate of active material 303. Accordingly, the first main surface, the second main surface, the first lateral surface, and the second lateral surface of the plate of active material 303 are not visible in FIG. 4.

FIG. 4 illustrates the plate of active material 303 having exposed lateral surfaces 304, 306. FIG. 4 also illustrates exposed lateral surfaces of the first metallic main surface layer 204, the second metallic main surface layer 206, the first lateral surface metal layer 208, and the second lateral surface metal layer 210. Advantageously, the first lateral surface metal layer 208 and the second lateral surface metal layer 210 always electrically connect the first metallic main surface layer 204 and the second metallic main surface layer 206 (that is, a “wrap-around” metallization of the plate of active material 303).

FIG. 5 illustrates the metallized plate of active material 302 after the bonding of an acoustic impedance matching layer 402 to its second metallic main surface layer 206 to form an intermediate assembly. Optionally, if this is provided in the acoustic stack structure, one or a plurality of additional acoustic impedance matching layers 404 may also be bonded to acoustic impedance matching layer 402. Although this is not shown in FIG. 5, the acoustic stack may also comprise an impedance dematching layer which forms an acoustic mirror—bonded to the first metallic main surface layer 204 of the plate of active material 302.

Acoustic impedance matching layer 402 and the acoustic impedance matching layer(s) 404 are preferably made of a particle-filled polymer material to obtain the desired acoustic properties (for example, of speed of sound), or graphite layers.

The bonding of the acoustic impedance matching layer 402 to the second metallic main surface layer 206 is preferably performed by adhesive bonding by depositing a thin layer of glue therebetween and by polymerizing it by thermal treatment (from room temperature to 100° C.) and under a pressure applied to the stack. According to a variant, the bonding is obtained by a molecular bonding process (“wafer bonding”).

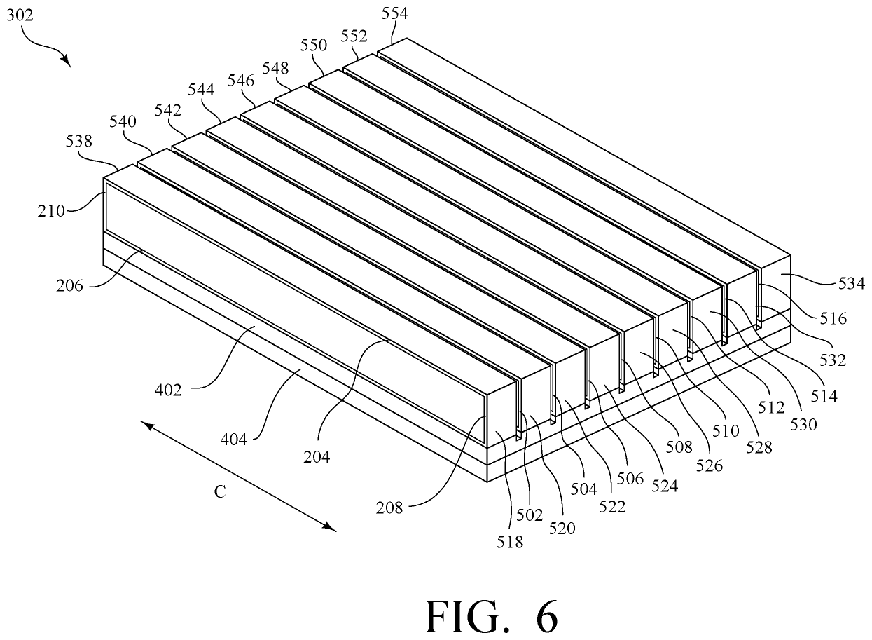

FIG. 6 illustrates the intermediate assembly after the full cutting of the metallized plate of active material 302 (that is, the full cutting across a thickness of the metallized plate of active material 302) in a first direction (that is, a C direction as indicated by the directional arrows designated with reference “C”) to form a first set of kerfs 502, 504, 506, 508, 510, 512, 514, 516. The first set of kerfs 502, 504, 506, 508, 510, 512, 514, 516 forms in the second metallic main surface layer 206 a set of column electrodes at the interface of the second metallic main surface layer 206 and of acoustic impedance matching layer 402, and forms in the first lateral surface metal layer 208 a first set of column electrode connectors 518, 520, 522, 524, 526, 528, 530, 532, 534 which electrically connect the column electrodes to the first metallic main surface layer 204. Correspondingly, the first set of kerfs 502, 504, 506, 508, 510, 512, 514, 516 also forms in the second lateral surface metal layer 210 a second set of column electrode connectors 538, 540, 542, 544, 546, 548, 550, 552, 554. The first set of column electrode connectors 518, 520, 522, 524, 526, 528, 530, 532, 534 and the second set of column electrode connectors 538, 540, 542, 544, 546, 548, 550, 552, 554 electrically connect the column electrodes to the first metallic main surface layer 204. Acoustic impedance matching layer 402 is used to hold in place the fully cut metallized plate of active material 302.

According to a variant, the cutting depth of the first set of kerfs 502, 504, 506, 508, 510, 512, 514, 516 may also extend through impedance matching layers 402 and 404 (only a partial cutting of impedance matching layer 402 is shown in FIG. 6). This depth extension is necessary if impedance matching layer 402 or impedance matching layers 402 and 404 are electrically conductive to avoid a short-circuit between column electrodes. In this case, the entire stack is previously bonded to a temporary mechanical hold substrate, which is then removed after the last transducer manufacturing step.

The first direction (that is, the C direction) is shown in FIG. 6 as being the width direction, without for this to be limiting. The only significant limit to the first direction it that it makes the first set of kerfs 502, 504, 506, 508, 510, 512, 514, 516 form in the second metallic main surface layer 206 the first set of column electrodes at the interface of the second metallic main surface layer 206 and of acoustic impedance matching layer 402, and form in the first lateral surface metal layer 208 the first set of column electrode connectors 518, 520, 522, 524, 526, 528, 530, 532, 534.

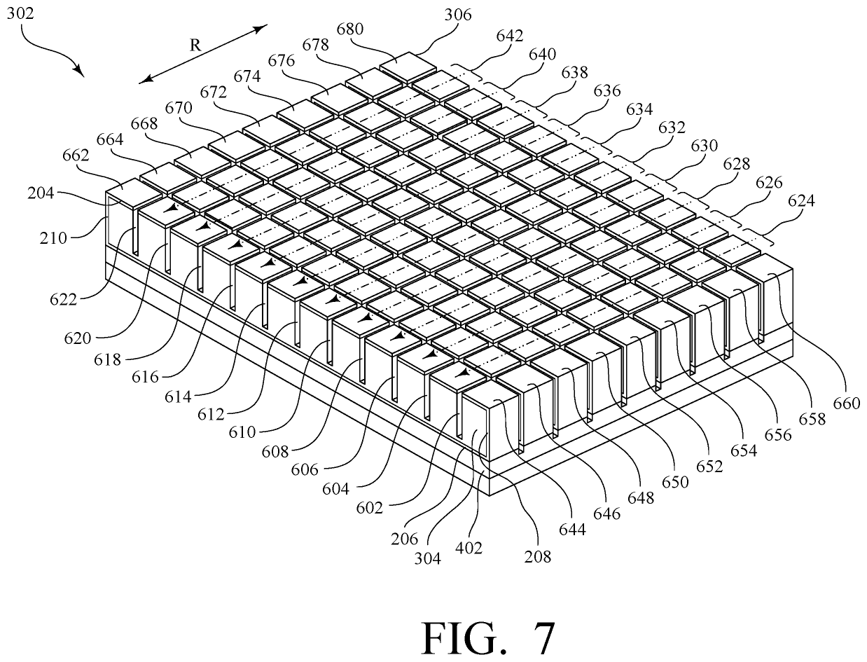

FIG. 7 illustrates the intermediate assembly after a partial cutting of the metallized plate of active material 302 (that is, by cutting less than the thickness of the plate of active material 303) in a second direction (that is, an R direction, as indicated by the directional arrows designated with reference “R”) to form a second set of kerfs 602, 604, 606, 608, 610, 612, 614, 61, 618, 620, 622. The cutting depth has to be smaller than the thickness of the plate of active material 303 to preserve the electric continuity of the set of column electrodes at the interface of the second metallic main surface layer 206 and of acoustic impedance matching layer 402. The second set of kerfs 602, 604, 606, 608, 610, 612, 614, 616, 618, 620, 622 forms in the first metallic main surface layer 204 rows of row electrodes 624, 626, 628, 630, 632, 634, 636, 638, 640, 642, a first external row of column electrode contacts 644, 646, 648, 650, 652, 654, 656, 658, 660, and a second external row of column electrode contacts 662, 664, 668, 670, 672, 674, 676, 678, 680. The first external row of column electrode contacts 644, 646, 648, 650, 652, 654, 656, 658, 660 is electrically connected respectively to the first set of column electrode connectors 518, 520, 522, 524, 526, 528, 530, 532, 534 (FIG. 6), and the second external row of column electrode contacts 662, 664, 668, 670, 672, 674, 676, 678, 680 is electrically connected respectively to the second set of column electrode connectors 538, 540, 542, 544, 546, 548, 550, 552, 554 (FIG. 6). Thus, the previously-described “wrap-around” metallization of the plate of active material 303 (FIG. 4) enables the set of column electrodes (that is, the C array electrodes) at the interface of the second metallic main surface layer 206 and of the acoustic impedance matching layer 402 to be electrically connected respectively to the first external row of column electrode contacts 644, 646, 648, 650, 652, 654, 656, 658, 660 and to the second external row of column electrode contacts 662, 664, 668, 670, 672, 674, 676, 678, 680. Advantageously, the first external row of column electrode contacts 644, 646, 648, 650, 652, 654, 656, 658, 660 and the second external row of column electrode contacts 662, 664, 668, 670, 672, 674, 676, 678, 680 form specific interconnect areas for C electrodes on opposite edges of the first metallic main surface layer 204 and on the same side of the metallized plate of active material 302 as the rows of row electrodes 624, 626, 628, 630, 632, 634, 636, 638, 640, 642 (that is, the R array electrodes), forming a single interconnection layer both for the R array electrodes and for the C array electrodes.

It should be noted that a single one among the first external row of column electrode contacts 644, 646, 648, 650, 652, 654, 656, 658, 660 and the second external row of column electrode contacts 662, 664, 668, 670, 672, 674, 676, 678, 680, and only one corresponding connector among the first set of column electrode connectors 518, 520, 522, 524, 526, 528, 530, 532, 534 and the second set of column electrode connectors 538, 540, 542, 544, 546, 548, 550, 552, 554 (FIG. 6) is necessary to contact the C array electrodes. However, providing both the first external row of column electrode contacts 644, 646, 648, 650, 652, 654, 656, 658, 660 and the second external row of column electrode contacts 662, 664, 668, 670, 672, 674, 676, 678, 680 (and the corresponding first set of column electrode connectors 518, 520, 522, 524, 526, 528, 530, 532, 534 and second set of column electrode connectors 538, 540, 542, 544, 546, 548, 550, 552, 554 (FIG. 6)) provides more fan-out options for the interconnection layer, as will be discussed hereafter.

It should further be noted that, in the intermediate assembly shown in FIG. 7, the partial cutting of the metallized plate of active material 302 to form the second set of kerfs 602, 604, 606, 608, 610, 612, 614, 61, 618, 620, 622 also forms in the plate of active material 302 the transducer elements in an array of rows and columns (that is, the pillars shown in FIG. 7). It will be understood by those skilled in the art that the order of the full cutting and of the partial cutting of the metallized plate of active material 302 may be inverted, so that the partial cutting is performed before the full cutting of the metallized plate of active material 302. Thus, it will be understood that the order of the full cutting and of the partial cutting is of no importance to form in the plate of active material 302 transducer elements in an array of rows and columns (that is, the pillars shown in FIG. 7).

The second direction (R direction) is oriented according to an angle of orientation relative to the first direction (C direction). As shown, the angle of orientation is orthogonal or of 90 degrees. Advantageously, with an orthogonal angle of orientation, the second set of kerfs 602, 604, 606, 608, 610, 612, 614, 61, 618, 620, 622 may be limited to extend through the exposed lateral surfaces 34, 306 of the plate of active material 302. However, the angle of orientation may comprise other angles preferably between, and including, 80 degrees and 90 degrees, so that the second set of kerfs 602, 604, 606, 608, 610, 612, 614, 61, 618, 620, 622 extends through the exposed lateral surfaces 304, 306 of the plate of active material 302. As an example, a small difference (<10°) with respect to a 90° angle may be advantageous for certain imaging applications where certain imaging directions are preferred. The difference with respect to a 90° angle may not be too significant, since the second set of kerfs 602, 604, 606, 608, 610, 612, 614, 61, 618, 620, 622 would no longer extend through the exposed lateral surfaces 304, 306 of the plate of active material 302 (unless the lateral dimensions of the transducer are increased).

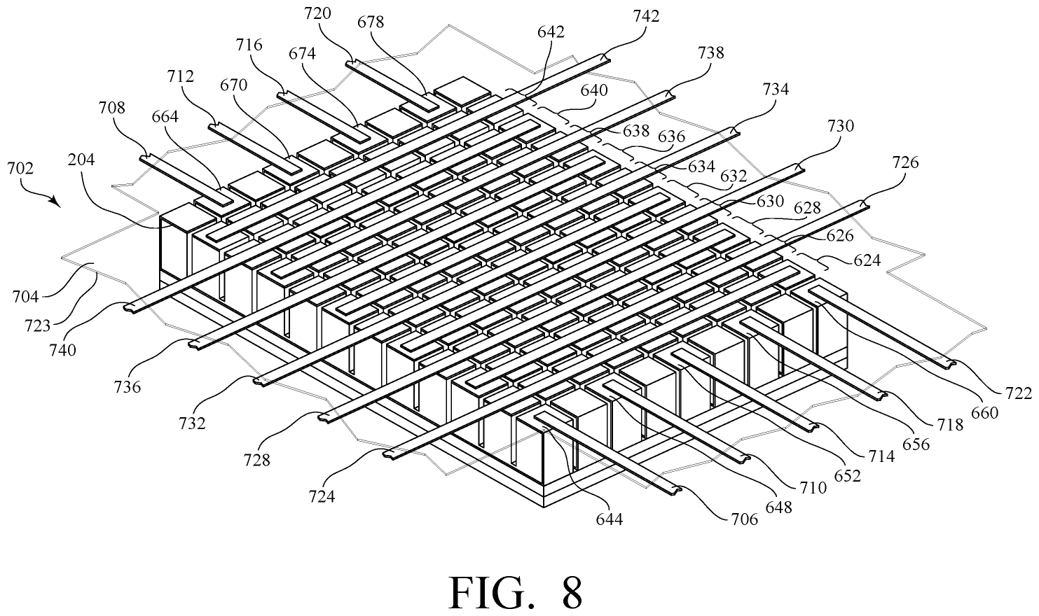

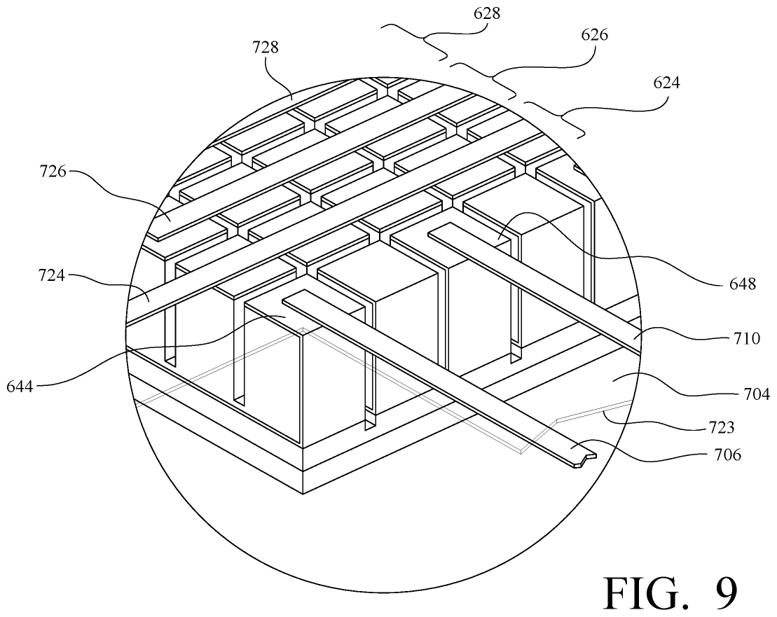

FIG. 8 and FIG. 9 illustrate the intermediate device obtained after the bonding of an interconnection layer 702 to the first metallic main surface layer 204. Interconnection layer 702 is preferably a printed circuit board, and may be a flex printed circuit board, a rigid printed circuit board, or flex-rigid printed circuit board. Flex-rigid printed circuit boards are boards using a combination of the rigid and flex board technologies in an application.

Interconnection layer 702 comprises a substrate 704 (that is, a support layer) which is formed of an electrically-insulating material. For simplification and clarity, substrate 704 is shown as being clear or transparent, but it will be understood by those skilled in the art that substrate 704 may also be translucent or opaque.

Interconnection layer 702 also comprises a first set of conductive tracks 706, 710, 714, 718, 722 and a third set of conductive tracks 708, 712, 716, 720 on an inner surface 723 of substrate 704. The inner surface 723 of substrate 704 is directed towards the first metallic main surface layer 204 on the metallized plate of active material 302. Each track of the first set of conductive tracks 706, 710, 714, 718, 722 is in electrical communication with a respective column electrode contact of the first external row of column electrode contacts 644, 648, 652, 656, 660. Each track of the third set of conductive tracks 708, 712, 716, 720 is in electrical communication with a respective column electrode contact of the second external row of column electrode contacts 664, 670, 674, 678. FIG. 8 illustrates a connection of the first set of conductive tracks 706, 710, 714, 718, 722 and the third set of conductive tracks 708, 712, 716, 720 with respective contacts both of the first external row of column electrode contacts 644, 648, 652, 656, 660 and of the second external row of column electrode contacts 664, 670, 674, 678, which are in electrical communication with respective column electrodes of the set of the column electrodes at the interface of the second metallic main surface layer 206 and of acoustic impedance matching layer 402. However, it should be noted that all connections could be formed either of the first external row of column electrode contacts 644, 646, 648, 650, 652, 654, 656, 658, 660 (see FIG. 7), or of the second external row of column electrode contacts 662, 664, 668, 670, 672, 674, 676, 678, 680 (see FIG. 7) and always be in electrical communication with respective column electrodes of the set of column electrodes at the interface of the second metallic main surface layer 206 and of acoustic impedance matching layer 402.

The first set of conductive tracks 706, 710, 714, 718, 722 forms a first interconnect fan-out of interconnection layer 702, and the third set of conductive tracks 708, 712, 716, 720 forms a third interconnect fan-out of interconnection layer 702. Advantageously, the alternation of the tracks from the first set of conductive tracks 706, 710, 714, 718, 722 and the third set of conductive tracks 708, 712, 716, 720 allows a larger interval between conductive tracks since the pitch between conductive tracks is double the pitch of the transducer columns. However, as previously described, the electrical connections with the set of column electrodes at the interface of the second metallic main surface layer 206 and of acoustic impedance matching layer 402 may be formed on a same side of the metallized plate of active material 302 (that is, a non-interdigitated configuration) in a single fan-out of the interconnection layer (that is, a “first unique set” of conductive tracks including the first set of conductive tracks 706, 710, 714, 718, 722 and the third set of conductive tracks 708, 712, 716, 720).

Interconnection layer 702 also comprises a second set of conductive tracks 724, 728, 732, 736, 740 and a fourth set of conductive tracks 726, 730, 734, 738, 742 on the inner surface 723 of substrate 704. Each track of the second set of conductive tracks 724, 728, 732, 736, 740 and of the fourth set of conductive tracks 726, 730, 734, 738, 742 is in electrical communication with a respective row among the rows of row electrodes 624, 626, 628, 630, 632, 634, 636, 638, 640, 642.

The second set of conductive tracks 724, 728, 732, 736, 740 forms a second interconnect fan-out of interconnection layer 702, and the fourth set of conductive tracks 726, 730, 734, 738, 742 forms a fourth interconnect fan-out of interconnection layer 702. Advantageously, the alternation of the tracks from the second set of conductive tracks 724, 728, 732, 736, 740 and the fourth set of conductive tracks 726, 730, 734, 738, 742 forms an interdigitated configuration of the rows and allows a larger interval between conductive tracks since the pitch between conductive tracks is double the pitch of the transducer rows. However, it should be noted that the electrical connections with the rows of row electrodes 624, 626, 628, 630, 632, 634, 636, 638, 640, 642 may be formed on the same side of the metallized plate of active material 302 (that is, a non-interdigitated configuration) in a single fan-out of the interconnection layer (that is, a “second unique set” of conductive tracks including the second set of conductive tracks 724, 728, 732, 736, 740 and the fourth set of conductive tracks 726, 730, 734, 738, 742).

Accordingly, all the interconnects of the row and column elements of the row-column ultrasound transduction device are formed on the first side 723 of substrate 704 without needing vias or a folding of said substrate. However, it should be noted that one or both of the first set of conductive tracks 706, 710, 714, 718, 722 and the third set of conductive tracks 708, 712, 716, 720 may be on an outer surface of substrate 704 and connected to respective contacts of the first external row of column electrode contacts 644, 646, 648, 650, 652, 654, 656, 658, 660 (see FIG. 7), or of the second external row of column electrode contacts 662, 664, 668, 670, 672, 674, 676, 678, 680 (see FIG. 7) by using vias, as will be described hereafter.

In certain embodiments, each track of the first set of conductive tracks 706, 708, 710, 712, 714, 716, 718, 720, 722 and of the second set of conductive tracks 724, 726, 728, 730, 732, 734, 736, 738, 740, 742 are in electrical contact with respectively the first external row of column electrode contacts 644, 648, 652, 656, 660, with the second external row of column electrode contacts 664, 670, 674, 678, and with row electrodes 624, 626, 628, 630, 632, 634, 636, 638, 640, 642, for example via a bonding material, for example a conductive glue or adhesive, or by an anisotropic conductive film.

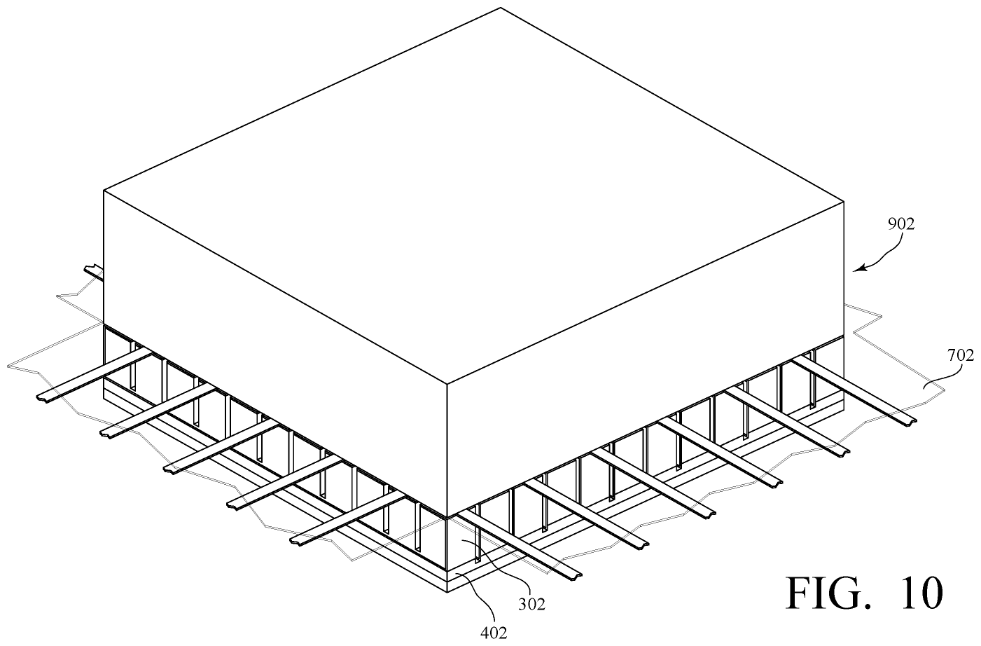

FIG. 10 illustrates an assembled RCA ultrasound transduction device comprising the metallized plate of active material 302, acoustic impedance matching layer 402, and interconnection layer 702, as previously described, and a set of backing layers 902 bonded to interconnection layer 702. The bonding of the set of backing layers 902 to interconnection layer 702 is preferably performed by adhesive bonding, by deposition of glue therebetween and a method of polymerization by thermal treatment (from room temperature to 100° C.) and under an applied stack pressure.

In an embodiment, backing layers 902 are passive materials having acoustic damping or absorption properties such as an epoxy resin, either as a solid material or comprising absorbing fillers or air bubbles. In another embodiment, the backing layers comprise an integrated circuit having the object of integrating in compact fashion the transduction device with its driver circuit. In this case, one or a plurality of layers used as an acoustic mirror (or “dematching layer”) are interposed between the integrated circuit and the rest of the transduction device to avoid for the integrated circuit to cause parasitic reflections of the ultrasound wave towards the transducer. Advantageously, the layers forming the acoustic mirror are conductive, the main inner surface is directly in electrical contact with the electrodes of the RCA transduction device without using interconnection layer 702, the main outer surface is in contact with electric pads on the integrated circuit.

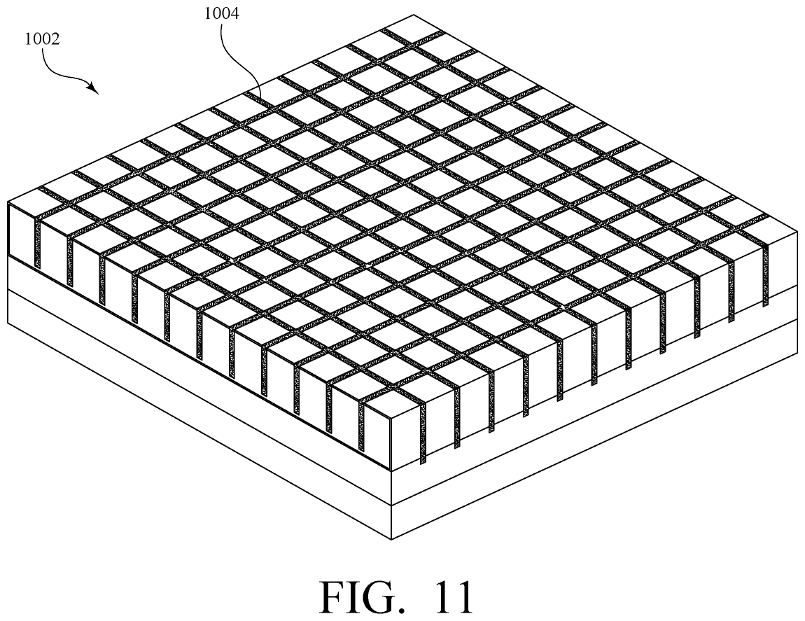

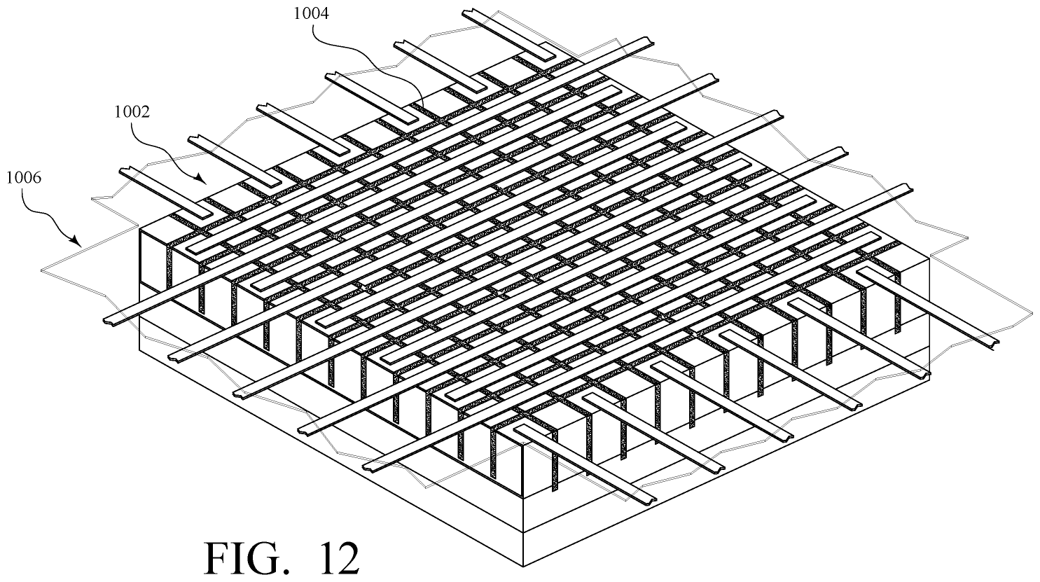

FIG. 11 and FIG. 12 illustrate an alternative embodiment, similar to the embodiment illustrated in FIG. 7 and in FIG. 8, of an intermediate assembly after cutting of a metallized plate of active material 102 to create kerfs, where the kerfs are filled with, for example, a polymer material 1004 before the bonding of an interconnection layer 1006. The polymer material ensures a mechanical decoupling between the transducer elements and may include fillers or air bubbles.

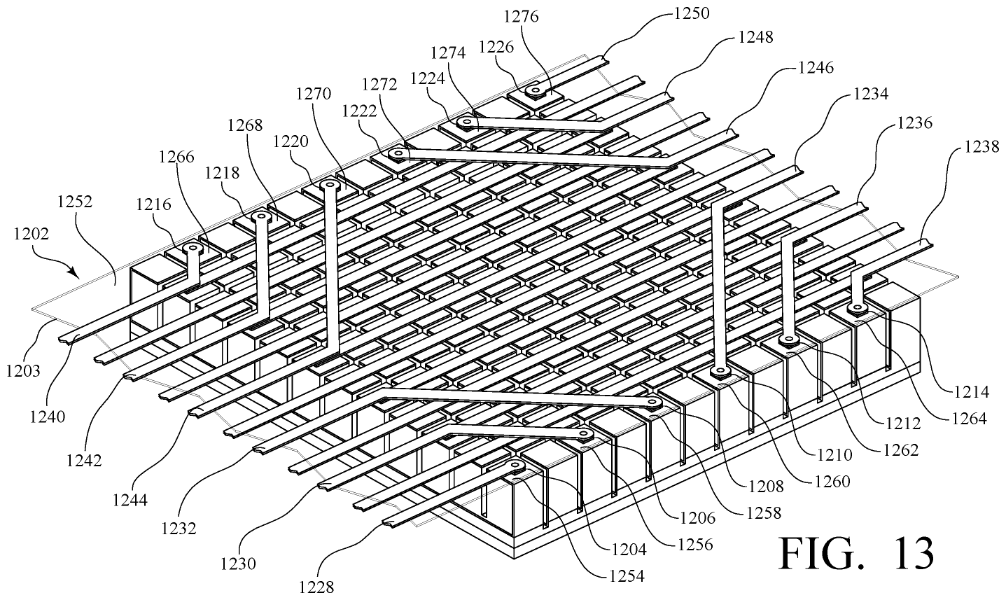

FIG. 13 shows another alternative embodiment, where interconnection layer 1202 comprises an insulating substrate 1203 provided with interconnect fan-outs on the two main surfaces of insulating substrate 1203. The interconnect fan-out of the second set of conductive tracks 724, 726, 728, 730, 732, 734, 736, 738, 740, 742 of interconnection layer 702 is unchanged in the case of interconnection layers 1202. Interconnection layer 1202 is provided with a first set of electrical contact pads 1204, 1206, 1208, 1210, 1212, 1214, located on the main inner surface of insulating substrate 1203, and in respective electrical contact among a first external row of column electrode contacts 1254, 1256, 1258, 1260, 1262, 1264; and with a second set of electrical contact pads 1216, 1218, 1220, 1222, 1224, 1226 located on the main inner surface of insulating substrate 1203, and in respective electrical contact among a second external row of column electrode contacts 1266, 1268, 1270, 1272, 1274, 1276. Vias, that is, vertical electrical connections crossing insulating substrate 1203, respectively connect a first set of electrical contact pads 1204, 1206, 1208, 1210, 1212, 1214, to a first set of conductive tracks 1228, 1230, 1232, 1234, 1236, 1238; and a second set of electrical contact pads 1216, 1218, 1220, 1222, 1224, 1226 respectively to a second set of conductive tracks 1240, 1242, 1244, 1246, 1248, 1250. This embodiment is particularly advantageous in that it enables to double the number of conductive tracks per surface area unit of interconnection layer 1202. The set of electric interconnect tracks 1234, 1236, 1236, 1246, 1248, 1250, forms a first electrical interconnect fan-out on a first lateral surface of the RCA transduction device. Similarly, the set of electric interconnect tracks 1228, 1230, 1232, 1240, 1242, 1242, forms a second electrical interconnect fan-out on a second lateral surface of the RCA transduction device opposite to the first lateral surface. In an alternative implementation, the first and the second electrical interconnect fan-out come out through a same lateral surface of the RCA transduction device. However, it should further be noted that if the electrical connections with the set of column electrodes are formed on a same lateral surface of the metallized plate of active material 302, and the electrical connections with the rows of row electrodes are formed on the same lateral surface of the metallized plate of active material 302, then the number of fan-outs may be decreased to one by placing the respective conductive tracks on opposite sides of substrate 1203 and by using vias and connection pads.

FIGS. 14, 15, and 16 illustrate an alternative embodiment using metallized vias 1301 vertically crossing the plate of active material 102 and electrically connecting the column electrodes coating the lower surface 106 of the plate of active material 102 (formed in lower surface metal layer 206) to upper surface metal layer 204.

Metallized vias 1301 are for example arranged at the end of the columns of the ultrasound transducer. As an example, each column comprises two metallized vias 1301 respectively arranged at the two ends of the column. As an example, each column may comprise a single metallized via arranged at one end of the column. As a variant, each column may comprise more than two metallized vias 1301.

In this example, metallized vias 1301 functionally replace column electrode connectors 518, 520, 522, 524, 526, 528, 530, 532, 534, and column electrode connectors 538, 540, 542, 544, 546, 548, 550, 552, 554, which electrically connect the column electrodes to the first metallic main surface layer 204.

Thus, in this example, the lateral surfaces of the plate of active material 102 at the ends of the columns may be non-metallized.

The transducer-forming method is similar to what has been previously described in relation with FIGS. 2 to 13. Only the differences with respect to the methods of FIGS. 2 to 13 are detailed hereafter.

The method of FIGS. 14, 15, and 16 differs from the examples described in relation with FIGS. 2 to 13 in that, in the example of FIGS. 14, 15, and 16, through openings, for example cylindrical, are formed in the raw plate of active material 102 at the desired locations of the vias 1301 of the future RCA ultrasound transducer.

The openings are for example formed by photolithography and etching, or by any other adapted method of forming of through openings in a raw plate of active material.

A wrap-around metallization is then formed to obtain a metallized raw plate of active material 202, similarly to what has been previously described in relation with FIG. 3. During this step, the through openings are filled with metal or have their sides coated with metal, forming metallized vias 1301.

FIG. 14 is a perspective view of the metallized raw plate of active material 202 obtained at the end of this step. In this drawing, metallized vias 1301 are shown in transparency for illustration purposes.

The metallized raw plate of active material 202 is then cut to individualize a metallized plate of active material 302 at the desired dimensions of the ultrasound transducer, similarly to what has been described hereabove in relation with FIG. 4.

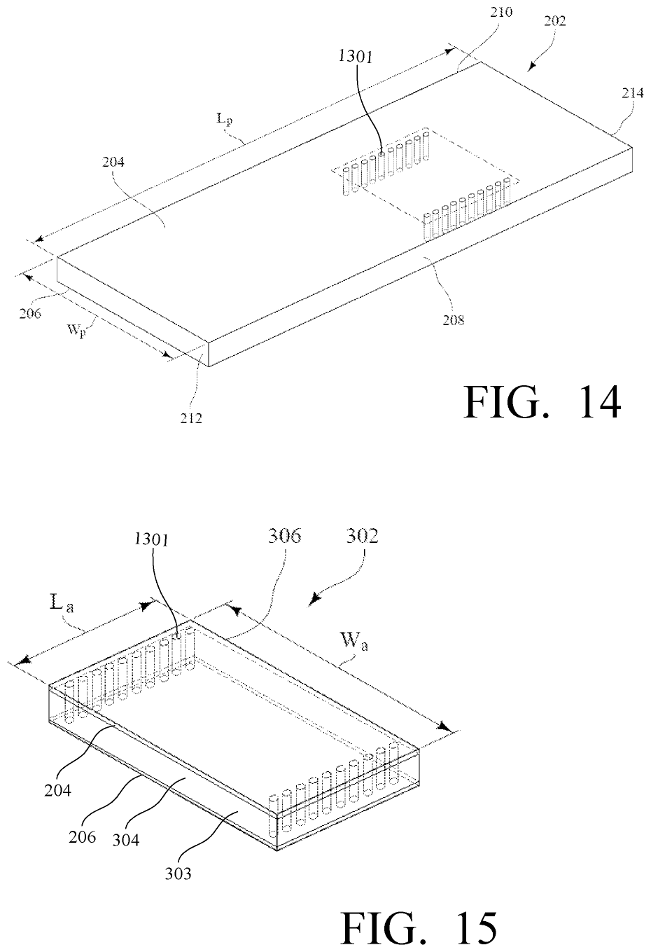

FIG. 15 is a perspective view schematically illustrating the metallized plate of active material 302 obtained at the end of the cutting step.

It should be noted that in this example, the dimensions of the metallized plate of active material 302 may be smaller than the dimensions of the metallized raw plate of active material 202. In particular, the width Wa of the metallized plate of active material 302 may be smaller than the width Wp of the metallized raw plate of active material 202, and the length La of the metallized plate of active material 302 may be smaller than the length Lp of the metallized raw plate of active material 202. In this case, none of the lateral surfaces of plate 302 is metallized. In other words, the lateral surfaces of the plate of active material 303 are all exposed, as illustrated in FIG. 15.

The next steps of the method are for example identical or similar to what has been previously described in relation with FIGS. 5, 6, 7, 8, 9, 10, and, possibly, 11, 12 and/or 13.

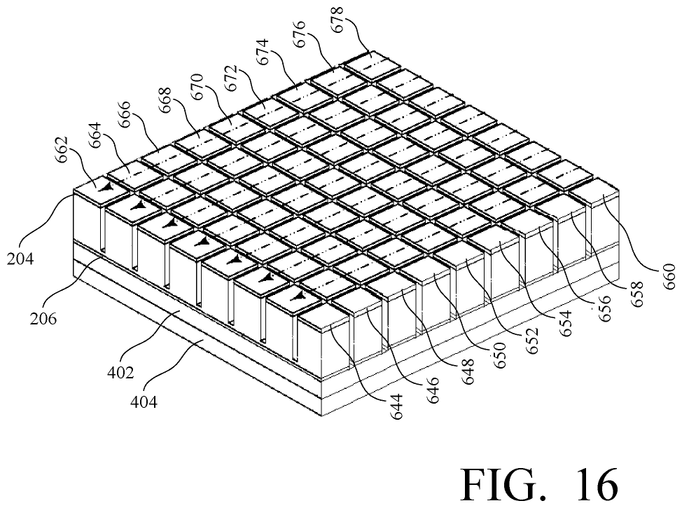

FIG. 16 illustrates the structure obtained at an intermediate stage of the method, corresponding to the structure obtained at the end of the steps described in relation with FIGS. 5, 6, and 7.

An advantage of the embodiment described in relation with FIGS. 14, 15, and 16 is that it enables to form transducers having lateral dimensions smaller than those of the raw plate, that is, a plurality of active plates may be cut in both lateral directions. In this case, the lateral sides of the active plate are no longer metallized to form a wrap-around such as described in the examples of FIGS. 2 to 13. The function of electrical connection of the column electrodes located on the side of the lower surface of the active plate to metallizations located on the upper surface side of the active plate is then ensured by metallized vias 1301.

It should be understood that different details of the object described herein may be modified without departing from the framework of the object described and claimed herein. Further, the previous description only has an illustration purpose and is by no way limiting. As an example, the numbers of rows and of columns of the shown ultrasound array transducers (RCA) have been simplified as an illustration, while real ultrasound transducers may comprise many more elements in each direction, for example 64, 128, 256, or more transducer elements in each of the row and column directions.

What is claimed is:

1. A row-column addressing array ultrasound transduction device, comprising:
a metallized plate of active material comprising a plate of active material having a first main surface and a second main surface opposite to the first main surface, a first metallic main surface layer on the first main surface, and a second metallic main surface layer on the second main surface;
an acoustic impedance matching layer bonded to the second metallic main surface layer;
a first set of parallel cutting notches oriented in a first direction, said notches of the first set extending across the entire thickness of the metallized plate of active material and along the entire length of the metallized plate of active material in the first direction, and individualizing the second metallic main surface layer into a set of column electrodes;
a second set of parallel cutting notches oriented along a second direction different from the first direction, said notches of second set extending across the entire thickness of the first metallic main surface layer and across at least part of the thickness of the plate of active material, and along the entire length of the metallized plate of active material in the second direction, and individualizing the first metallic main surface layer into a set of rows of row electrodes and a first external row of column electrode contacts, each column electrode contact of the first external row of column electrode contacts being electrically connected to a respective column electrode of the set of column electrodes; and
an interconnection layer comprising a substrate of an electrically-insulating material, a first set of conductive tracks on the substrate, each track of the first set of conductive tracks being in electrical contact with a respective column electrode contact of the first external row of column electrode contacts, and a second set of conductive tracks on the substrate, each track of the second set of conductive tracks being in electrical communication with a respective row among the rows of row electrodes.

2. The device according to claim 1, wherein each column electrode contact of the first external row of column electrode contacts is electrically connected to the respective corresponding column electrode of the set of column electrodes by a respective column electrode connector of a first set of column electrode connectors formed in a first lateral surface metal layer covering a first lateral surface of the plate of active material, the column electrode connectors of the first set of column electrode connectors being individualized by the cutting notches of the first set of cutting notches.

3. The device according to claim 1, wherein each column electrode contact of the first external row of column electrode contacts is electrically connected to the respective corresponding column electrode of the set of column electrodes by a metallized via vertically crossing the plate of active material.

4. The device according to claim 1, wherein the substrate of the interconnection layer comprises an inner surface, wherein the first set of conductive tracks and the second set of conductive tracks are on the inner surface of the substrate, and wherein the first set of conductive tracks forms a first interconnect fan-out of the interconnection layer, and the second set of conductive tracks forms a second interconnect fan-out of the interconnection layer.

5. The device according to claim 1,
wherein the substrate of the interconnection layer comprises an inner surface, an outer surface, vias respectively aligned with the first external row of column electrode contacts, and respective via connectors in the vias;
wherein the first set of conductive tracks is on the outer surface of the substrate and is in electrical communication with respective column electrode contacts of the first external row of column electrode contacts by means of via connectors, and the second set of conductive tracks is on the inner surface of the substrate; and
wherein the first set of conductive tracks and the second set of conductive tracks form a first interconnect fan-out of the interconnection layer.

6. The device according to claim 1,
wherein the second set of parallel cutting notches further individualizes a second external row of column electrode contacts, each column electrode contact of the first external row of column electrode contacts being electrically connected to a respective column electrode of the set of column electrodes.

7. The device according to claim 1, wherein the first and second directions are orthogonal.

8. The device according to claim 1, wherein the first set of cutting notches and the second set of cutting notches are filled with a polymer material.

9. The device according to claim 1, wherein the interconnection layer is a flex printed circuit board.

10. The device according to claim 1, further comprising a set of backing layers bonded to the interconnection layer.

11. A method of manufacturing a row-column addressing array ultrasound transduction device, the method comprising:
the provision of a metallized raw plate of active material comprising a plate of active material having a first main surface and a second main surface opposite to the first main surface, a first metallic main surface layer on the first main surface, and a second metallic main surface layer on the second main surface, the metallized raw plate of active material having a width Wp and a length Lp;
the cutting of the metallized raw plate of active material to individualize a metallized plate of active material from the metallized raw plate of active material;
the bonding of an acoustic impedance matching layer to the second metallic main surface layer of the metallized plate of active material;
the full cutting of the metallized plate of active material in a first direction to form a first set of parallel cutting notches, the first set of cutting notches forming in the second metallic main surface layer a set of column electrodes;
the partial cutting of the metallized plate of active material in a second direction to form a second set of parallel cutting notches, the second set of cutting notches forming in the first metallic main surface layer rows of row electrodes and a first external row of column electrode contacts, each column electrode contact of the first external row of column electrode contacts being electrically connected to a respective column electrode of the set of column electrodes;
the provision of an interconnection layer comprising a substrate of an electrically-insulating material, a first set of conductive tracks on the electrically-insulating substrate, a second set of conductive tracks on the electrically-insulating substrate;
the connection of each track of the first set of conductive tracks so that it is in electrical communication with a respective column electrode contact of the first external row of column electrode contacts; and
the connection of each track of the second set of conductive tracks in electrical contact with a respective row among the rows of row electrodes.

12. The method according to claim 11, wherein each column electrode contact of the first external row of column electrode contacts is electrically connected to the respective corresponding column electrode of the set of column electrodes by a respective column electrode connector of a first set of column electrode connectors formed in a first lateral surface metal layer of the metallized plate of active material, covering a first lateral surface of the plate of active material, the column electrode connectors of the first set of column electrode connectors being individualized by the cutting notches of the first set of cutting notches.

13. The method according to claim 12, wherein the metallized plate of active material has a length La smaller than the length Lp of the metallized raw plate of active material and a width Wa equal to the width Wp of the metallized raw plate of active material.

14. The method according to claim 11, wherein each column electrode contact of the first external row of column electrode contacts is electrically connected to the respective corresponding column electrode of the set of column electrodes by a metallized via of the metallized plate of active material, vertically crossing the plate of active material.

15. The method according to claim 14, wherein the metallized plate of active material has a length La smaller than the length Lp of the metallized raw plate of active material and a width Wa smaller than the width Wp of the metallized raw plate of active material.
